(19) United States
(12) Patent Application Publication
(10) Pub. No.: US 2005/0137620 A1
(43) Pub. Date: Jun. 23, 2005

(54) Balloon catheter retrieval device

(75) Inventor: Yousef F. Alkhatib, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SciMed Life Systems, Inc.

(21) Appl. No.: 10/745,716

(22) Filed: Dec. 22, 2003

(51) Int. Cl. A61M 29/00

(52) U.S. Cl. 606/194; 604/103.05

(57) ABSTRACT

An intracorporeal assembly includes an elongated shaft having a proximal portion and an opposing distal portion, a balloon disposed on the distal portion, and a tubular member disposed about the elongated shaft. The tubular member can slide from the proximal portion to the distal portion of the elongate shaft to surround a portion of the balloon.

### FIELD OF THE INVENTION

The present invention is related to the field of medical devices. In particular, the present invention is related to the field of catheters.

### BACKGROUND OF THE INVENTION

Heart and vascular disease are major problems in the United States and throughout the world. Conditions such as atherosclerosis result in blood vessels becoming blocked or narrowed. This blockage can result in lack of oxygenation of the heart, which has significant consequences, since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire so that the balloon is positioned adjacent a stenotic lesion. The balloon is then inflated, and the restriction of the vessel is opened.

A wide variety of balloon catheters and angioplasty balloons exist, each with certain advantages and disadvantages. A balloon procedure can have an associated risk in that the balloon may become lodged within the patient's vasculature. If the balloon is lodged within the patient's vasculature or caught on an implanted stent, the operator must either apply additional force to the catheter, subjecting the device to a higher risk of failure or the balloon must be removed surgically, subjecting the patient to the risks of surgery. There is an ongoing need for balloon catheter retrieval devices.

### SUMMARY OF THE INVENTION

Generally, the present invention relates to an intracorporeal retrieval device useful for a variety of applications including, for example, retrieval of a balloon catheter.

In one embodiment, an intracorporeal assembly includes an elongated shaft having a proximal portion and an opposing distal portion, a balloon disposed on the distal portion, and a tubular member disposed about the elongated shaft. The tubular member can slide from the proximal portion to the distal portion of the elongate shaft to surround a portion of the balloon.

In another embodiment, a method includes placing an elongate shaft having a proximal portion outside a subject vasculature and a distal portion within the subject vasculature. A balloon disposed on the distal portion is expanded within the subject vasculature. A tubular member disposed about the elongate shaft is moved from the proximal portion of the elongate shaft to the distal portion of the elongate shaft to surround a portion of the balloon. The tubular member, balloon and elongate shaft are removed from the subject vasculature.

The above summary of the present invention is not intended to describe each disclosed embodiment or every implementation of the present invention. The Figures and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side elevation view of a balloon catheter deployed within a subject vasculature;

FIG. 1A is a cross-sectional view of the catheter shaft of FIG. 1. taken along line 1A-1A;

FIG. 2 is a perspective view of a balloon catheter and tubular member assembly;

FIG. 3 is a perspective view of a proximal end of a tubular member showing an exemplary gripping surface;

FIG. 4 is a top view of an exemplary eyeloop;

FIG. 5 is a front elevation view of the eyeloop shown in FIG. 4;

FIG. 6 is a side elevation view of the eyeloop shown in FIG. 4;

FIG. 7 is a side elevation view of the balloon catheter shown in FIG. 1 lodged within a subject vasculature;

FIG. 8 is a side elevation view of the balloon catheter shown in FIG. 1 being dislodged within a subject vasculature; and

FIG. 9 is a side elevation view of the balloon catheter shown in FIG. 1 being removed from within a subject vasculature with the tubular member.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE PREFERRED EMBODIMENTS

The intracorporeal retrieval device and method for using the intracorporeal retrieval device of the present invention are believed to be applicable to a variety of applications where retrieval of intracorporeal devices is desired, for example, retrieval of a balloon catheter from vasculature. While the present invention is not so limited, an appreciation of various aspects of the invention will be gained through a discussion of the examples provided below.

The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

Also, while the tubular members illustrated in the Figures have generally circular cross sections, this is not a necessary part of the present invention, and the tubular members are merely shown as such for purposes of simplicity in illustration.

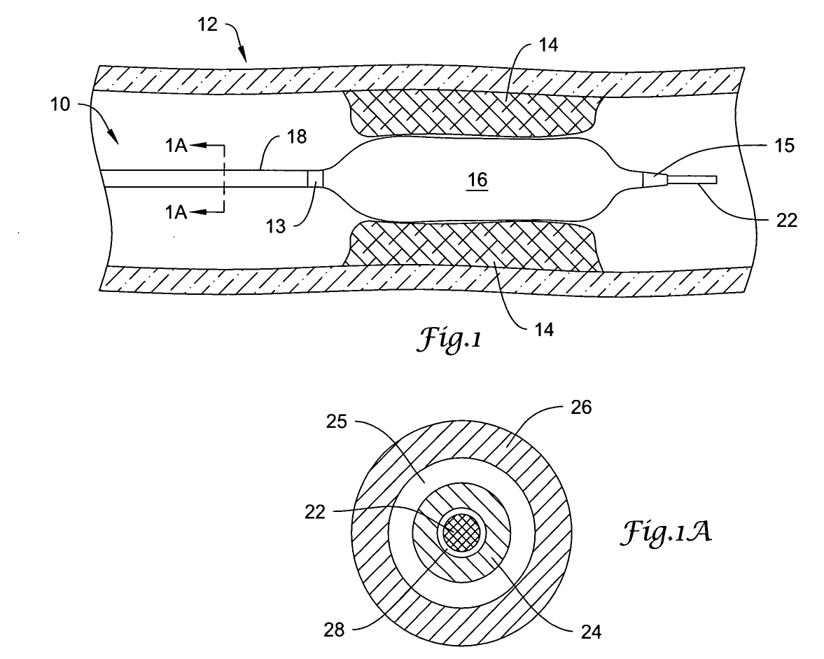

Angioplasty techniques have been shown to be effective for at least some intravascular interventions. FIG. 1 illustrates an example angioplasty catheter 10 positioned in a blood vessel 12 adjacent an intravascular lesion 14. Catheter 10 may include a balloon 16 coupled to a catheter shaft 18. In general, catheter 10 may be advanced over a guidewire 22 through the vasculature to a target area. Balloon 16 can then be inflated to expand lesion 14. The target area may be within any suitable peripheral or cardiac location.

Balloon 16 may be made from typical angioplasty balloon materials including polymers such as polyethylene terephthalate (PET), polyetherimide (PEI), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, Marlex high-density polyethylene, Marlex low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro(propyl vinyl ether) (PFA), other suitable materials, or mixtures, combinations, copolymers thereof, polymer/metal composites, and the like. In some embodiments, it may be desirable to use high modulus or generally stiffer materials so as to reduce balloon elongation. The above list of materials includes some examples of higher modulus materials. Some other examples of stiffer materials include polymers blended with liquid crystal polymer (LCP) as well as the materials listed above. For example, the mixture can contain up to about 5% LCP.

Shaft 18 may be a catheter shaft, similar to typical catheter shafts. FIG. 1A is a cross-sectional view of the catheter shaft shown in FIG. 1. For example, shaft 18 may include an inner tubular member 24 and outer tubular member 26. Tubular members 24/26 may be manufactured from a number of different materials. For example, tubular members 24/26 may be made of metals, metal alloys, polymers, metal-polymer composites or any other suitable materials. Some examples of suitable metals and metal alloys include stainless steel, such as 304V, 304L, and 316L stainless steel; nickel-titanium alloy such as linear-elastic or super-elastic Nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; or other suitable material. Some examples of suitable polymers include those described above in relation to balloon 16. Of course, any other suitable polymer may be used without departing from the spirit of the invention. The materials used to manufacture inner tubular member 24 may be the same as or be different from the materials used to manufacture outer tubular member 26.

Tubular members 24/26 may be arranged in any appropriate way. For example, in some embodiments, inner tubular member 24 can be disposed coaxially within outer tubular member 26. According to these embodiments, inner and outer tubular members 24/26 may or may not be secured to one another along the general longitudinal axis of shaft 18. Alternatively, inner tubular member 24 may follow the inner wall or otherwise be disposed adjacent the inner wall of outer tubular member 26. Again, inner and outer tubular members 24/26 may or may not be secured to one another. For example, inner and outer tubular members 24/26 may be bonded, welded (including tack welding or any other welding technique), or otherwise secured at a bond point. In some embodiments, the bond point may be generally disposed near the distal end of shaft 18. However, one or more bond points may be disposed at any position along shaft 18. The bond may desirably impact, for example, the stability and the ability of tubular members 24/26 to maintain their position relative to one another. In still other embodiments, inner and outer tubular member 24/26 may be adjacent to and substantially parallel to one another so that they are non-overlapping. In these embodiments, shaft 18 may include an outer sheath that is disposed over tubular members 24/26.

Inner tubular member 24 may include an inner lumen 28. In at least some embodiments, inner lumen 28 is a guidewire lumen. Accordingly, catheter 10 can be advanced over guidewire 22 to the desired location. The guidewire lumen may extend along essentially the entire length of catheter shaft 18 so that catheter 10 resembles traditional “over-the-wire” catheters. Alternatively, the guidewire lumen may extend along only a portion of shaft 18 so that catheter 10 resembles “single-operator-exchange” or “rapid-exchange” catheters. Regardless of which type of catheter is contemplated, catheter 10 may be configured so that balloon 16 is disposed over at least a region of inner lumen 28. In at least some of these embodiments, inner lumen 28 (i.e., the portion of inner lumen 28 that balloon 16 is disposed over) may be substantially coaxial with balloon 16.

Shaft 18 may also include an inflation lumen 30 that may be used, for example, to transport inflation media to and from balloon 16. The location and position of inflation lumen 30 may vary, depending on the configuration of tubular members 24/26. For example, when outer tubular member 26 is disposed over inner tubular member 24, inflation lumen 30 may be defined within the space between tubular members 24/26. Moreover, depending on the position of inner tubular member 24 within outer tubular member 26, the shape of lumen 30 (i.e., the shape adjacent shaft 18) may vary. For example, if inner tubular member 24 is attached to or disposed adjacent to the inside surface of outer tubular member 26, then inflation lumen 30 may be generally half-moon in shape; whereas if inner tubular member 24 is generally coaxial with outer tubular member 26, then inflation lumen 30 may be generally ring-shaped or annular in shape. It can be appreciated that if outer tubular member 26 is disposed alongside inner tubular member 24, then lumen 30 may be the lumen of outer tubular member 26 or it may be the space defined between the outer surface of tubular members 24/26 and the outer sheath disposed thereover.

Balloon 16 may be coupled to catheter shaft 18 in any of a number of suitable ways. For example, balloon 16 may be adhesively or thermally bonded to shaft 18. In some embodiments, a proximal waist 32 of balloon 16 may be bonded to shaft 18, for example, at outer tubular member 26, and a distal waist 34 may be bonded to shaft 18, for example, at inner tubular member 24. The exact bonding positions, however, may vary. It can be appreciated that a section of proximal waist 32 may not have sections 36 extending therefrom in order for suitable bonding between balloon 16 and outer tubular member 30.

In addition to some of the structures described above, shaft 18 may also include a number of other structural elements, including those typically associated with catheter shafts. For example, shaft 18 may include a radiopaque marker coupled thereto that may aid a user in determining the location of catheter 10 within the vasculature. In addition, catheter 10 may include a folding spring (not shown) coupled to balloon 16, for example, adjacent proximal waist 32, which may further help in balloon folding and refolding. A description of a suitable folding spring can be found in U.S. Pat. No. 6,425,882, which is incorporated herein by reference.

Every angioplasty procedure has a risk that the balloon will become lodged or stuck within the subject vasculature. The balloon can get caught on a stent or artificial object that was placed within the subject or the balloon can get caught on the lesion or other vasculature architecture. In addition, during deflation the balloon may not rewrap to its original size prior to inflation. In all these cases, the balloon must be extracted by either applying additional forces to the balloon catheter and risking product failure within the subject vasculature or extracting the balloon via surgery. However, in accordance with the present invention, a tubular assembly may be used to dislodge stuck balloon catheters from the subject vasculature.

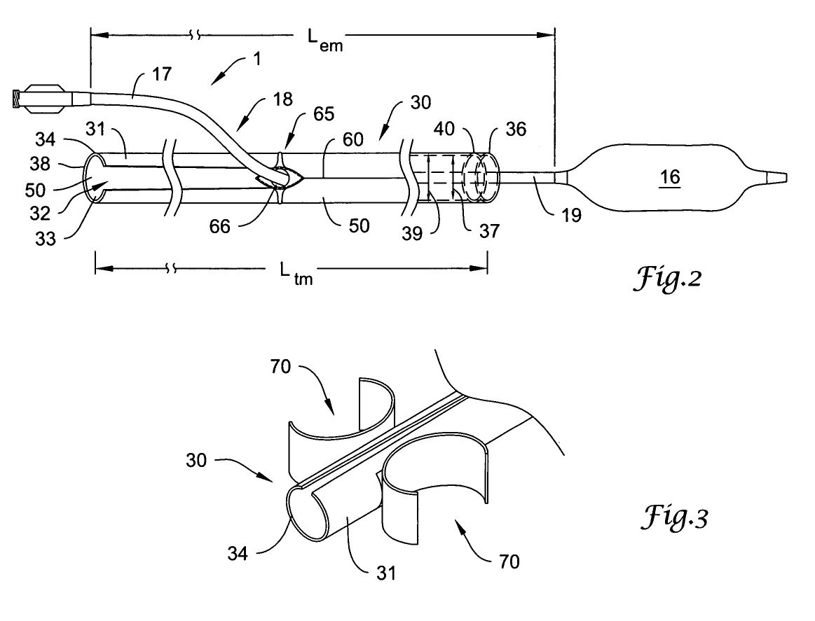

FIG. 2 is a perspective view of a balloon catheter 1 and tubular member 30 assembly. The tubular member 30 can be disposed about the elongate shaft 18 of the balloon catheter 1. The tubular member 30 can slide from a proximal portion 17 of the elongate shaft 18 to a distal portion 19 of the elongate shaft 18 and surround a portion of the balloon 16. The balloon catheter 1 may be any useful type of balloon catheter such as, for example, a cardiovascular balloon catheter or a peripheral vascular balloon catheter. The balloon catheter 1 may or may not be loaded with a stent.

The elongate shaft 18 is an elongate member 18 having a length Lₑₘ defined by the distance between an end of a proximal portion 17 and an opposing end of a distal portion 19. The elongate member length Lₑₘ can be any length that allows the balloon 16 to reach the target within the subject vasculature such as, for example, 10 to 300 cm, 50 to 200 cm or 100 to 150 cm.

The tubular member 30 has a lumen 32 defined by a tubular member inner surface 33 having an inner diameter 37. The tubular member lumen 32 can be any operable diameter to encompass a balloon catheter shaft. The tubular member lumen 32 or inner diameter can be any useful size such as, for example, 0.01 to 1 inch, 0.02 to 0.5 inch, or 0.02 to 0.2 inch.

The tubular member 30 has a length Lₜₘ defined by the distance between a tubular member proximal end 34 and an opposing tubular member distal end 36. The tubular member length Lₜₘ can be any operable length to reach the balloon within the subject vasculature such as, for example, 10 to 300 cm, 50 to 200 cm or 100 to 150 cm.

The tubular member length Lₜₘ can be equal to or greater than the elongate member length Lₑₘ. Alternatively, the tubular member length Lₜₘ can be equal to or less than the elongate member length Lₑₘ.

The tubular member 30 has a wall 38 defined by the distance between the tubular member inner diameter 37 (inner surface 33) and the tubular member outer diameter 39 (outer surface 31). The wall 38 can be any operable thickness that provides enough rigidity to be able to push the tubular member 30 to the lodged balloon, but is not so thick as to hinder the tubular member 30 flexibility to follow the curvature of the subject vasculature. The tubular member wall thickness can be, for example, 0.001 to 0.1 inch, or 0.001 to 0.05 inch. In an illustrative embodiment, the tubular member 30 is large enough to slide over a conventional balloon catheter 1 but small enough to fit within a conventional guide catheter.

The tubular member 30 can include a radiopaque marker 40 disposed at or near the tubular member distal end 36. The radiopaque marker 40 aids a user in determining the location of the tubular member distal end 36 within the vasculature. The radiopaque marker can be formed of any radiopaque material.

The tubular member 30 can be formed of a lubricious material or include a coating 50 of a lubricious material in the tubular member inner surface 33 or tubular member outer surface 31 or on both the inner surface 33 and the outer surface 31.

The tubular member 30 can include a slit 60 extending through the tubular member wall 38 along a portion of the tubular member length Lₜₘ, or along the entire tubular member length Lₜₘ. The slit 60 can begin at the tubular member proximal end 34 and end short of the tubular member distal end 36. The slit 60 can extend 50%, 60%, 70%, 80% or 90% of the total tubular member length Lₜₘ.

In one embodiment, the tubular member 30 can include an eyeloop 65 for engaging the balloon catheter 1. The eyeloop 65 defines an eyeloop opening 66 extending through the eyeloop 65. The eyeloop 65 can be any desired shape that provides for the operation of the device such as, for example, circular, elliptical, and the like. The balloon catheter 1 or elongate shaft 18 can extend through the eyeloop opening 66. The eyeloop 65 can be slidably engaged along the tubular member slit 60. An exemplary eyeloop 65 is further described below. The tubular member 30 can include a gripping surface 70 described below.

A gripping surface 70 can be disposed at or near the proximal end 34 of the tubular member 30. The gripping surface 70 provides an enhanced user interface with the tubular member 30 by allowing the user to easily manipulate the tubular member 30 within the subject vasculature. The gripping surface 70 allows the user to easily advance the tubular member 30 forward toward the stuck balloon or to easily reverse retrieval of the balloon.

FIG. 3 shows an illustrative embodiment of the gripping surface 70. In this embodiment, the gripping surface 70 can be exemplified by a pair of double-sided wings set on the tubular member outer surface 31. The pair of double-sided wings are configured to allow human fingers to fit inside the wings cradle to control advancement and/or withdrawal of the tubular member 30 within the vasculature.

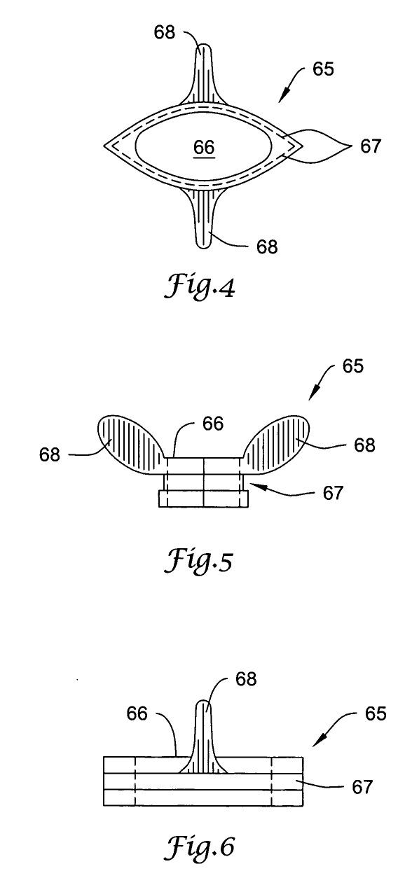

FIG. 4 is a top view of an exemplary eyeloop 65. FIG. 5 is a front elevation view of the eyeloop 65 shown in FIG. 4. FIG. 6 is a side elevation view of the eyeloop 65 shown in FIG. 4. In the illustrative embodiment, the eyeloop 65 can include an eyeloop opening 66, a slot 67 and wings 68. A balloon catheter can be preloaded into the eyeloop opening 66 prior to a procedure. The slot 67 engages each side of the slit 60, holding each side of the slit 60 in place. When the tubular member 30 is needed to retrieve a balloon, the tubular member 30 can be advanced distally forward along the balloon catheter 1 elongate shaft 18. At the same time, the eyeloop 65 can be slide in a proximal direction engaging more of the tubular member 30 on the elongate shaft. In the illustrative embodiment, the eyeloop 65 can be moved via wings 68 placed on the eyeloop 65. The wings 68 can be configured to allow a user to manipulate the position of the eyeloop 65 along the length of the slit 60 with the user's fingers.

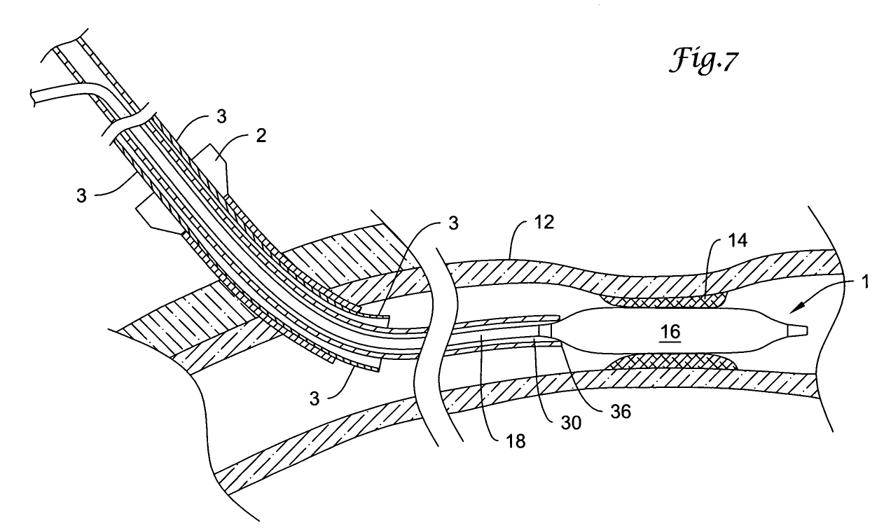

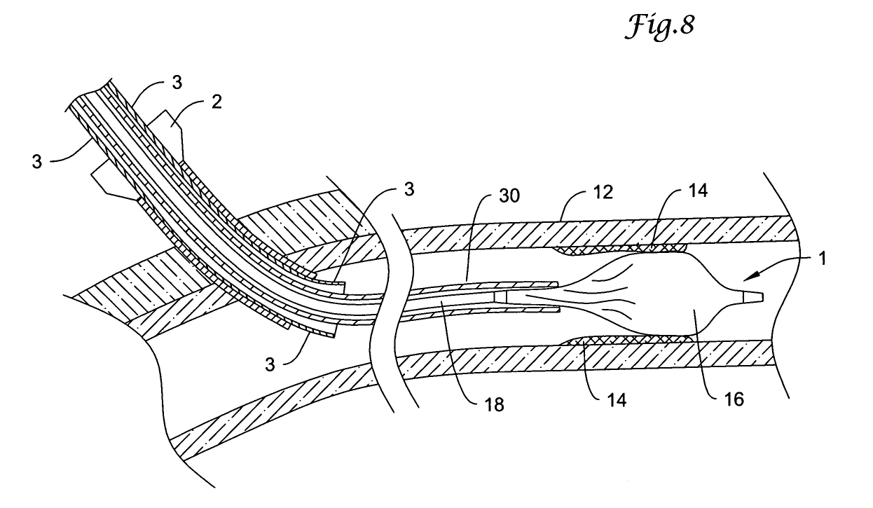

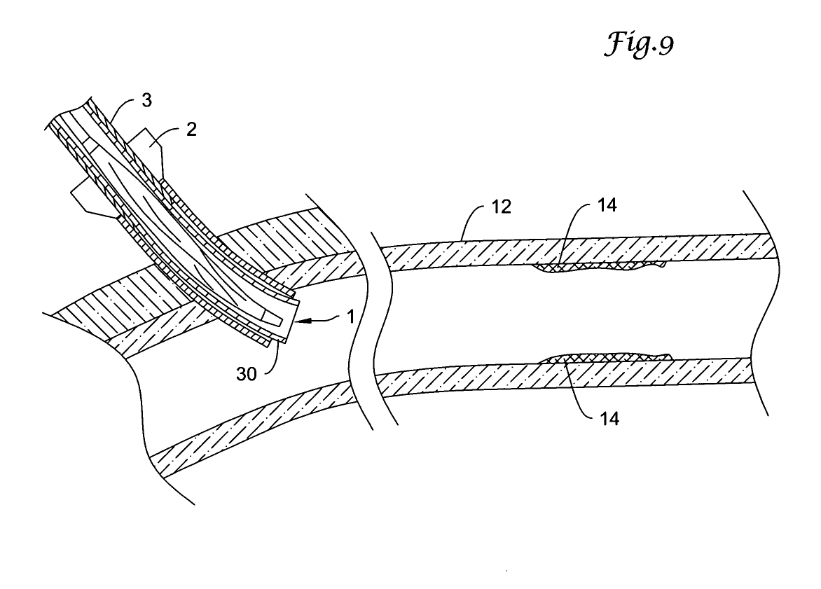

FIG. 7 is a side elevation view of the balloon catheter 1 shown in FIG. 1 lodged within a subject vasculature 12. FIG. 8 is a side elevation view of the balloon catheter 1 shown in FIG. 1 being dislodged within a subject vasculature 12. FIG. 9 is a side elevation view of the balloon catheter 1 shown in FIG. 1 being removed within a subject vasculature 12 with the tubular member 30. As shown in FIG. 7, the tubular member 30 can be advanced into a vessel 12 around the balloon catheter 1 elongate shaft 18 and within for example, an introducer sheath 2 and/or guide catheter 3. The tubular member 30 is advanced to the lodged balloon catheter 1 and surrounds a portion of the balloon 16. The tubular member distal end 36 can touch the balloon 16 or interface between the balloon 16 and vessel 12 or stent (not shown) and cause the balloon 16 to be dislodged from the vasculature 12 or stent and rewrap to allow for easy withdrawal from the vasculature 12.

During the above procedure, a proximal portion of the tubular member 30 can remain outside the vasculature 12. In one illustrative embodiment, the tubular member 30 can be pre-loaded onto the balloon catheter 1 prior to the procedure. A preloaded tubular member 30 can have a slit 60 extending along a portion of the tubular member length Lₜₘ as described above. The tubular member 30 can be advanced along the elongate member 18 to the stuck balloon 16. The tubular member “swallows” the balloon 16 and allows the tubular member 30, balloon 16, and elongate shaft 18 to be removed at the same time from the vasculature 12.

In another illustrative embodiment, the tubular member 30 can be loaded onto the balloon catheter 1 elongate member 18 prior to the procedure or after the balloon 16 is placed within the vasculature. The tubular member can have a slit 60 extending along the entire length of the tubular member 30. The tubular member 30 can be loaded onto the elongate member 18 via the slit 60 and advanced along the elongate member 18 to the stuck balloon 16. The tubular member 30 “swallows” the balloon 16 and allows the tubular member 30, balloon 16, and elongate shaft 18 to be removed at the same time from the vasculature 12.

In another illustrative embodiment, the tubular member 30 without a slit 60 can be loaded onto the balloon catheter 1 elongate member 18 after the balloon 16 is placed within the vasculature by advancing the tubular member 30 along the elongate member 18 to the stuck balloon 16. The tubular member “swallows” the balloon 16 and allows the tubular member 30, balloon 16, and elongate shaft 18 to be removed at the same time from the vasculature 12.

The present invention should not be considered limited to the particular examples described above, but rather should be understood to cover all aspects of the invention as fairly set out in the attached claims. Various modifications, equivalent processes, as well as numerous structures to which the present invention can be applicable will be readily apparent to those of skill in the art to which the present invention is directed upon review of the instant specification.

1. An intracorporeal assembly comprising:
a) an elongated shaft having a proximal portion and an opposing distal portion;
b) a balloon disposed on the distal portion; and
c) a tubular member disposed about the elongated shaft such that the tubular member can slide from the proximal portion to the distal portion of the elongate shaft to surround a portion of the balloon.

2. The intracorporeal assembly according to claim 1, wherein the tubular member defines a tubular member lumen having a tubular member length defined by the distance between a tubular member proximal end and a tubular member distal end and a tubular member wall defined by a distance between a tubular member inner diameter and a tubular member outer diameter, wherein the tubular member wall has a tubular member wall inner surface and a tubular member wall outer surface.

3. The intracorporeal assembly according to claim 2, wherein the tubular member comprises a radiopaque marker disposed near the tubular member distal end.

4. The intracorporeal assembly according to claim 2, wherein the elongate member has an elongate member length equal to or less than the tubular member length.

5. The intracorporeal assembly according to claim 2, further comprising a lubricious coating disposed on the tubular member.

6. The intracorporeal assembly according to claim 2, further comprising a lubricious coating disposed on the tubular member wall inner surface and the tubular member wall outer surface.

7. The intracorporeal assembly according to claim 2, further comprising a slit extending through the tubular member wall along a portion of the tubular member length.

8. The intracorporeal assembly according to claim 2, further comprising a slit extending through the tubular member wall along a distal portion of the tubular member length.

9. The intracorporeal assembly according to claim 2, further comprising a slit extending through the tubular member wall along a proximal portion of the tubular member length.

10. The intracorporeal assembly according to claim 2, further comprising a slit extending through the tubular member wall along an entire tubular member length.

11. The intracorporeal assembly according to claim 3, further comprising an eyeloop slidably engaged along the tubular member slit, wherein the eyeloop defines a eyeloop opening extending through the eyeloop.

12. The intracorporeal assembly according to claim 11, wherein the elongated shaft extends through the eyeloop opening.

13. The intracorporeal assembly according to claim 2, further comprising a gripping surface disposed on the tubular member near the proximal end.

14. The intracorporeal assembly according to claim 1, further comprising a guide catheter surrounding a portion of the tubular member disposed about the elongated shaft.

15. A method comprising:
a) placing an elongate shaft having a proximal portion outside a subject vasculature and a distal portion within the subject vasculature;
b) expanding a balloon that is disposed on the distal portion within the subject vasculature;
c) moving a tubular member that is disposed about the elongate shaft from the proximal portion of the elongate shaft to the distal portion of the elongate shaft to surround a portion of the balloon.
d) removing the tubular member, balloon and elongate shaft from the subject vasculature.

16. The method according to claim 15, wherein the expanding a balloon further comprises lodging the balloon within the subject vasculature.

17. The method according to claim 16, wherein the moving a tubular member dislodges the balloon from the subject vasculature.

18. The method according to claim 15, wherein the removing the tubular member, balloon and elongate shaft are removed from the subject vasculature at the same time.

19. The method according to claim 15, wherein the moving a tubular member comprises moving a tubular member disposed about the elongate member from the proximal portion outside a subject vasculature to the distal portion to surround a portion of the balloon within the subject vasculature.

20. The method according to claim 15, further comprising placing the tubular member on the elongate shaft before placing the elongate shaft within the subject vasculature.

21. The method according to claim 15, further comprising placing the tubular member on the elongate shaft after placing the elongate shaft within the subject vasculature.

22. The method according to claim 15, wherein the moving a tubular member comprises moving a tubular member disposed about the elongate member from the proximal portion outside a subject vasculature to the distal portion to surround a portion of the balloon and collapse the balloon within the subject vasculature.

23. The method according to claim 15, wherein the moving a tubular member further comprises sliding an eyeloop slidably engaged in a longitudinal slit along a length of the tubular member in a proximal direction as the tubular member is moved in a distal direction along the elongate member, wherein the eyeloop defines a eyeloop opening extending through the eyeloop and the elongate member extends through the eyeloop opening.

24. The method according to claim 15, wherein the moving a tubular member further comprises moving a tubular member that is disposed about the elongate shaft and within a guide catheter from the proximal portion of the elongate shaft to the distal portion of the elongate shaft to surround a portion of the balloon.
